(19) United States
(12) Patent Application Publication
(10) Pub. No.: US 2026/0130625 A1
(43) Pub. Date: May 14, 2026

(54) MINIMALLY INVASIVE GLUCOSE STATE SYSTEMS, DEVICES, AND METHODS

(71) Applicant: SynchNeuro, Inc., Philadelphia, PA (US)

(72) Inventors: Casey HALPERN, Philadelphia, PA (US); Emily MIRRO, Pacifica, CA (US)

(21) Appl. No.: 19/119,092

(22) PCT Filed: Oct. 10, 2023
(86) PCT No.: PCT/US2023/076474

§ 371 Date: Apr. 7, 2025

Related U.S. Application Data
(60) Provisional application No. 63/378,834, filed on Oct. 7, 2022.
(60) Provisional application No. 63/381,078, filed on Oct. 26, 2022.

(51) Int. Cl. A61B 5/00; A61B 5/145; A61B 5/291; A61B 5/372

(52) U.S. Cl. CPC A61B 5/4839; A61B 5/14532; A61B 5/291; A61B 5/372; A61B 5/6814; A61B 5/7246; A61B 5/742; A61B 2560/0223; A61B 2560/0468

(57) ABSTRACT

Glucose systems that include a minimally invasive scalp-worn device that includes first and second sensors. The systems are adapted to determine one or more glucose states based on sensed EEG signals using the scalp-worn device.

### CROSS REFERENCE TO RELATED APPLICATIONS

This application claims priority to U.S. Provisional Applications 63/378,834, filed Oct. 7, 2022, and 63/381,078, filed Oct. 26, 2022, the entire disclosures of which are incorporated by reference herein for all purposes.

### INCORPORATION BY REFERENCE

All publications and patent applications mentioned in this specification are herein incorporated by reference to the same extent as if each individual publication or patent application was specifically and individually indicated to be incorporated by reference.

This application incorporates by reference herein the following publications in their entireties for all purposes: U.S. Pat. Nos. 6,572,542; 8,118,741; and 11,020,035.

This application incorporates by reference herein WO/2023/183798A2 in its entirety and for all purposes.

Glucose monitors and/or insulin delivery devices and uses thereof, such as those described in, for example only, U.S. Pat. Nos. 9,585,607; 10,827,956; 11,744,943; 11,672,422; and 11,064,917 are incorporated by reference herein in their entireties for all purposes.

PCT publication WO/2023/034820A1 is incorporated by reference herein in its entirety for all purposes. The entire disclosure of U.S. Prov. App. No. 63/238,583, filed Aug. 30, 2021, to which the WO/2023/034820A1 application claims priority, is also incorporated by reference herein in its entirety for all purposes. The following article is also incorporated by reference herein in its entirety for all purposes: Huang, Y., Wang, J. B., Parker, J. J. et al. Spectro-spatial features in distributed human intracranial activity proactively encode peripheral metabolic activity. Nat Commun 14, 2729 (2023). https://doi. org/10.1038/s41467-023-38253-7.

### BACKGROUND OF THE DISCLOSURE

Blood glucose is one of the most important blood parameters to measure, as abnormal levels can cause severe complications. For example, blood glucose levels above or below standard levels can cause significant medical issues including medical emergencies.

Hypoglycemia, which requires medical attention, is a condition in which blood sugar (glucose) level is lower than the standard range, often considered less as a blood sugar of 70 milligrams per deciliter (mg/dL), or 3.9 millimoles per liter (mmol/L). While hypoglycemia may be related to diabetes management, hypoglycemia can occur and be associated with non-diabetes medical conditions and/or environments (e.g., ICU patients). Treatment of hypoglycemia may include high sugar food or drink (e.g., juice) and/or medicine in an attempt to raise the blood glucose level. Hypoglycemia, if left untreated, may lead to loss of consciousness and/or seizures.

Hyperglycemia is a condition when blood glucose levels are higher than the standard range, often considered above 180 milligrams per deciliter (mg/dL). Hyperglycemia, which is commonly linked to diabetes, occurs when the body has too little insulin (the hormone that transports glucose into the blood), or if the body can't use insulin properly. Hyperglycemia can, however, be associated with non-diabetes medical conditions and/or environments (e.g., ICU patients). If left untreated for long periods of time, hyperglycemia can cause damage to nerves, blood vessels, tissues and organs.

Existing technology can test current blood glucose levels, in near real-time. For example continuous glucose monitors (“CGM”) include a small sensor inserted under the skin, such as the abdomen or arm. The sensor measures the interstitial glucose (“ISG”) level, which is indicative of blood glucose levels. The sensor may test ISG every few minutes. A transmitter can wirelessly send the information to a monitor, which may be part of an insulin pump, or which may be a separate device, such as a smartphone or tablet. CGMs are essentially detecting existing or current blood glucose levels, and since ISG levels follow blood glucose levels by several minutes, the blood glucose levels provided by CGMs are in fact indicative of past blood glucose levels. Insulin pumps can automatically initiate insulin injection if glucose levels get too high. A threshold glucose level may be set (stored) in the CGM such that insulin may be delivered once the glucose level reaches the preset threshold. Individualized thresholds may be set for patients, and the thresholds may need to be re-set over time (currently at office visits), such as if changes in the patient's life necessitate resetting the threshold (e.g., going through puberty). Currently, the best insulin pumps for diabetes management achieve a peripheral glucose target range of 70 to 180 mg/dL <70% of the time, which is not ideal and still quite broad of a glycemic range.

Additionally, non-diabetic conditions and environments exist where it is desirable or necessary to know blood glucose levels. For example only, intensive care unit (“ICU”) patients, who may or may not be diabetic, typically have their blood levels frequently checked, typically using blood from a finger prick, which can be tested on a small strip with a meter that shows the glucose levels.

U.S. Pat. No. 6,572,542 describes utilizing ECG signals and EEG signals to determine if a hypoglycemic event is occurring or is imminent. While it may be helpful to determine if a glycemic event is occurring (e.g., such as with a CGM) or is imminent, there may be a need to be able to determine a real-time or near real-time glucose state generally, and/or forecast future blood glucose levels or risks of a future glycemic event in advance, including much further in advance than an imminent event.

### SUMMARY OF THE DISCLOSURE

One aspect of the disclosure is a method of forecasting a future glucose state of a subject, comprising: non-invasively sensing EEG signals from a behind the ear location on a scalp of a subject; inputting the EEG signals or processed EEG signals into a trained computer executable method trained to forecast a future glucose state of the subject; forecasting a future glucose state of the subject based at least partially on the non-invasively sensed EEG signal; and outputting instructions to initiate a communication based on and in response to the forecasted future glucose state. This aspect may include any other suitably combinable method or step herein.

One aspect of this disclosure is a computer-executable method, stored in a non-transitory media, adapted to, when executed by a processor, cause the performance of: receiving as input EEG data or processed EEG data non-invasively sensed from a behind the ear location on a scalp of a subject; forecasting a future glucose state of the subject based at least partially on the EEG data or processed EEG data; and initiating an output adapted to communicate information indicative of the forecasted future glucose state of the subject based on and in response to the forecasted future glucose state of the subject. This aspect may include any other suitably combinable method herein.

One aspect of this disclosure is a non-transitory, computer-readable storage media with instructions stored thereon and executable by a processor to perform a method, the method comprising: receiving as input EEG data or processed EEG data non-invasively sensed from a behind the ear location on a scalp of a subject; forecasting a future glucose state of the subject based at least partially on the EEG data or processed EEG data; and initiating an output adapted to communicate information indicative of the forecasted future glucose state of the subject based on and in response to the forecasted future glucose state of the subject. This aspect may include any other suitably combinable device, feature, and/or method herein.

One aspect of the disclosure is a method of forecasting a future glucose state of a subject, comprising: non-invasively sensing EEG signals with a single-channel EEG sensor on a scalp of a subject; inputting the EEG signals or processed EEG signals into a trained computer executable method trained to forecast a future glucose state of the subject; forecasting a future glucose state of the subject based at least partially on the non-invasively sensed EEG signal; and outputting instructions to initiate a communication based on and in response to the forecasted future glucose state. This aspect may include any other suitably combinable method herein.

One aspect of the disclosure is a computer-executable method adapted to, when executed by a processor, cause the performance of: receiving as input EEG data or processed EEG data non-invasively sensed with a single channel EEG sensor on a scalp of a subject; forecasting a future glucose state of the subject based at least partially on the EEG data or processed EEG data; and initiating an output adapted to communicate information indicative of the forecasted future glucose state of the subject based on and in response to the forecasted future glucose state of the subject. This aspect may include any other suitably combinable method herein.

One aspect of the disclosure is a system for forecasting a future glucose state of a subject, comprising: a wearable EEG sensor with a configuration and arrangement to be wearable on a scalp behind the ear; and a computer-executable method, stored in a non-transitory media of a personal device, and adapted to, when executed by a processor in the personal device, cause the performance of: receiving as input EEG data or processed EEG data non-invasively sensed from the EEG sensor; forecasting a future glucose state of the subject based at least partially on the EEG data or processed EEG data; and initiating an output adapted to communicate information indicative of the forecasted future glucose state of the subject based on and in response to the forecasted future glucose state of the subject. This aspect may include any other suitably combinable device, feature, and/or method herein.

One aspect of this disclosure is method of forecasting future glucose levels of a subject, comprising: sensing EEG signals from a subject with a behind-the-ear EEG device; processing the sensed EEG signals; with an application on a personal device, analyzing the processed EEG signals with a trained forecasting model and forecasting future glucose levels of the subject; and causing the personal device to visually present on a display information that is indicative of the forecasted future glucose levels. This aspect may include any other suitably combinable method herein.

One aspect of this disclosure is a computer executable method stored in a non-transitory memory of a personal device, comprising: receiving sensed EEG data from a subject or information indicative of sensed EEG data from a subject; analyzing the processed EEG signals with a trained forecasting model and forecasting future glucose levels of the subject; and causing the personal device to visually present on a display information that is indicative of the forecasted future glucose levels. This aspect may include any other suitably combinable method herein.

One aspect of the disclosure is a glucose forecasting system (GFS), comprising: a minimally invasive EEG device that includes first and second sensors, the EEG device sized, configured and adapted to be worn behind the ear of a subject and to sense EEG signals with the first and second sensors; and a personal device adapted to be in communication with the EEG device, the personal device further adapted to, receive and process the sensed EEG signals or information indicative of the sensed EEG signals, and analyze the processed EEG signals with a trained forecasting model to forecast future glucose levels of the subject. This aspect may include any other suitably combinable device, feature, and/or method herein. In this aspect, the personal device may be further adapted to visually present on a display of the personal device information that is indicative of the forecasted future glucose levels or a glucose state.

One aspect of the disclosure is a method of training a computer executable forecasting method for forecasting a future glucose state of a subject, comprising: non-invasively sensing training EEG signals from one or more subjects; processing the sensed training EEG signals; sensing or receiving training real-time or near real-time blood glucose levels from the one or more subjects, or information indicative of real-time or near real-time blood glucose levels; and correlating the sensed or the processed training EEG signals with the sensed or received training real-time or near real-time blood glucose levels or information that is indicative of real-time or near real-time blood glucose levels to train the method to forecast a future glucose state of an individual based on non-invasively sensed EEG signals from the individual.

One aspect of this disclosure is a computer executable method (e.g., an “App”) adapted to present forecasted future blood glucose levels or a glucose state, the computer executable method stored in a non-transitory memory, the method comprising: receiving as input extracranially-sensed EEG data or information indicative of extracranially-sensed EEG from a subject; causing a visual representation of the forecasted future blood glucose levels to be displayed on a display of a device. The visual representation may comprise a graph, with time on a first axis, with a second axis representing forecasted blood glucose levels or a glucose state. The visual representation may comprise textual information indicating a time or time range when blood glucose levels are likely to be undesirable.

### BRIEF DESCRIPTION OF THE DRAWINGS

FIG. 1 illustrates a system including an intracranial device.

FIG. 2 is a block diagram for exemplary controllers herein, aspects of which may be incorporated into any of the devices and systems herein.

FIG. 3 illustrates an exemplary flow chart for an exemplary process for predictively managing glucose levels.

FIG. 4 is an exemplary flow chart of an exemplary process for predicting glucose levels based on brain activity data.

FIG. 5 is a non-limiting example of a visual representation of forecasted blood glucose levels or a glucose state on a display of a device (optionally a personal device), such as a smartphone or other computing device.

FIG. 6 illustrates an exemplary glucose forecasting system including exemplary and non-limiting components.

FIG. 7 illustrate an exemplary continuous glucose forecasting system.

FIG. 8 illustrates a method that includes forecasting information indicative of a future glucose state using sensed interstitial glucose information (such as ISF glucose levels).

FIG. 9 illustrates a method that including managing a future glucose state using sensed interstitial glucose information (such as ISF glucose levels).

FIG. 10 illustrates a method that includes correlating or associating one or more aspects of sensed interstitial glucose information with one or more aspects of sensed EEG signals to create a correlation therebetween.

FIG. 11 illustrates an exemplary step of calibrating or recalibrating a glucose monitor using at least one of EEG data or information indicative of the sensed EEG data.

FIG. 12 illustrates an exemplary method that includes calibrating or recalibrating a blood glucose determination or blood glucose forecasting method.

FIG. 13 illustrates an exemplary bi-directional calibration method herein.

FIG. 14 illustrates an exemplary method that includes forecasting a future glucose state of a subject.

FIG. 15 illustrates an exemplary method of training a method to forecast a future glucose state of an individual.

FIG. 16 illustrates a method that includes forecasting a future glucose state of the subject based at least partially on a non-invasively sensed EEG signal.

FIG. 17 illustrates a portion of an exemplary personal device.

FIG. 18 illustrates a portion of an exemplary system.

FIG. 19 illustrates additional exemplary and optional components of any of the devices and systems herein (e.g., personal devices and/or systems that include a personal device).

### DETAILED DESCRIPTION

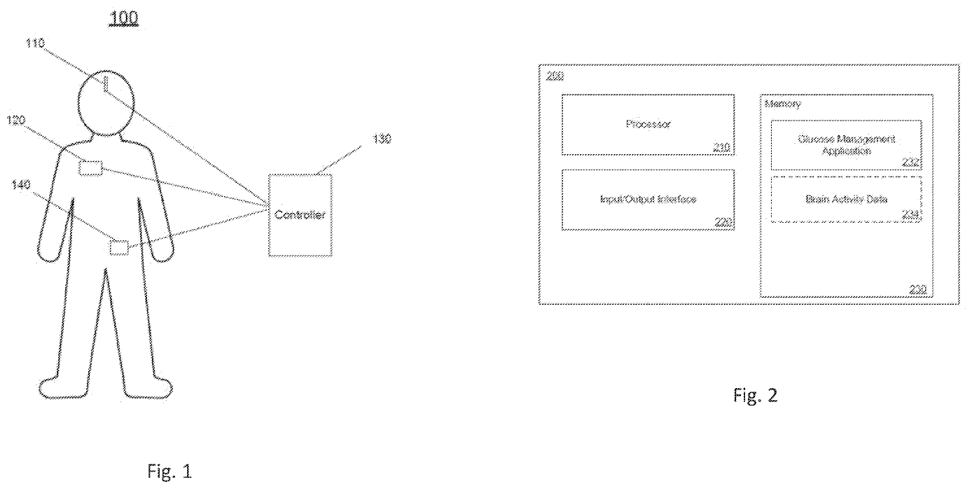

The disclosure is related to glucose forecasting systems and methods of use that include a behind-the-ear (or otherwise in close proximity to the ear), scalp-worn EEG device (EEG sensor). Much of the disclosure of PCT publication WO/2023/034820A 1 (published March 9, 2023) is expressly incorporated by reference below in paragraphs [0039]-[0055] and in FIGS. 1-4 herein, which may (but not necessarily) provide exemplary support and basis for one or more aspects of the glucose forecasting systems and methods of use herein that include a minimally invasive behind-the-ear scalp-worn EEG sensor. The entire disclosure of U.S. Prov. App. No. 63/238,583, filed Aug. 30, 2021, to which the WO/2023/034820A1 application claims priority, is also incorporated by reference herein in its entirety for all purposes. The entire following article, including but not limited to any methodology, is also incorporated by reference herein for all purposes, which is related to WO/2023/034820A1: Huang, Y., Wang, J. B., Parker, J. J. et al. Spectro-spatial features in distributed human intracranial activity proactively encode peripheral metabolic activity. Nat Commun 14, 2729 (2023). https://doi.org/10.1038/s41467-023-38253-7.

Metabolic syndromes and diabetes are increasingly prevalent health conditions, now affecting a broader range of ages in our global population. Specifically, the significant morbidity and mortality associated with diabetes have created a major toll on the healthcare system, including extensive costs, both personal and societal, in the form of medical expenditures for the disease itself and loss of workforce productivity from disability associated with disease progression. Hence, the ability to prevent development of diabetes and its subsequent complications is a high-impact area of public health for intervention. Additionally, eating behavior and physiologic regulation of the body's metabolic and weight balance entails a complex interplay of hormonal signaling and behaviors. In this context, close monitoring and control of blood glucose levels has been shown to be one of the best and most reliable methods to prevent complications of both hypo-and hyperglycemia.

Controlling blood glucose is important beyond the scope of diabetes, as both hyper-and hypoglycemia in hospitalized and critically-ill patients are associated with increased cost, length of stay, morbidity, and morality. Patients, especially those in intensive care units may suffer from stress-related hyperglycemia as a result of severe injuries and illnesses, e.g. traumatic brain injury (TBI), intracranial hemorrhage, stroke, subarachnoid hemorrhage (SAH), and many others. Conservative glycemic control has been associated with better outcomes in these patients.

Current iterations of continuous glucose monitors (CGMs) rely on interstitial glucose measurements as a proxy for blood glucose levels, which have an intrinsic lag time and are subject to interference by medications and extreme blood glucose values. Conventional CGMs are also unable to anticipate abnormal glucose levels; as a reactive modality, they can only respond to hypo or hyperglycemia once the abnormality has already occurred. In addition, patients with severe disease may have chronically elevated glucose levels that are refractory to conventional treatment options including continuous insulin infusion. Systems and methods described herein seek to rectify these limitations by predicting what a patient's glucose levels will be in the next several hours. Using this information, preemptive treatment can be delivered to the patient in order to avoid deleterious glucose levels from occurring.

In many embodiments, predictive glucose management (PGM) systems and methods described with respect to FIGS. 1-4 decode brain activity in order to predict the patient's future glucose levels. In a variety of embodiments, brain activity is measured using a non-invasive modality such as electroencephalography (EEG), functional near-infrared spectroscopy (fNIRS), magnetoencephalography (MEG), or any other modality as appropriate to the requirements of specific applications of embodiments herein. However, brain activity can be recorded using intracranial sensors if available, such as (but not limited to) deep brain stimulation (DBS) systems, or ECOG. In various embodiments, PGM systems are wearable or otherwise minimally invasive with respect to a patient's life outside of a clinical setting.

In numerous embodiments, systems and methods described herein involve closed-loop management of glucose levels, where preemptive treatment is provided to the patient to avoid hyper-or hypo-glycemia. For example, patients may be delivered long-acting insulin, an insulin analogue, and/or any other hyperglycemia control drug as appropriate to the requirements of specific applications in anticipation of future glucose changes. By way of further example, brain stimulation may be provided in order to perturb the glucose encoding network in the brain in order to alter blood glucose levels for the subsequent several hours. Brain stimulation may be provided by an already implanted DBS electrode depending on implantation location, any other type of implanted electrode, or via a non-invasive brain stimulation modality such as (but not limited to) transcranial magnetic stimulation (TMS), transcranial direct current stimulation (tDCS), transcranial focused ultrasound (tFUS), and/or any other modality as appropriate to the requirements of specific applications. In various embodiments, brain stimulation modalities may be utilized as an adjunct to insulin when the patient is refractory to standard treatments. PGM system architectures are discussed in further detail below.

PGM systems record and decode brain activity to estimate likely glucose levels of a patient in the next several hours. Typically, the predictions are accurate out for at least 2-8 hours, although depending on the patient and condition, this number may increase. In many embodiments, PGM systems provide these predictions to the patient and/or medical professionals. However, in various embodiments, PGM systems are further capable of closed-loop glucose level control by continuously predicting future glucose levels and altering treatment (e.g. drug delivery rate, brain stimulation, etc.) to avoid the predicted deleterious change in glucose level. In this way, PGMs can perform as an artificial pancreas system with superior glucose management capabilities. In some embodiments, patient and/or medical professional authorization is required before treatment is delivered and/or modified by the PGM.

FIG. 1 illustrates an example PGM system architecture in accordance with an embodiment. PGM system 100 includes a brain activity recorder 110. In the illustrated embodiment, brain activity recorder 110 is a deep brain stimulation system. However, as can readily be appreciated, any brain activity recorder can be used, including those that are non-invasive as discussed above. In some embodiments, the brain activity recorder is a wearable device, rather than an implanted device. PGM system 100 further includes a CGM 120. In many embodiments CGMs are used to continuously confirm the accuracy of interstitial blood glucose predictions, and may further act as a redundant warning modality. However, CGMs may not be present in all PGM systems as appropriate to the requirements of specific applications.

A controller 130 is communicatively coupled with the brain activity recorder 110, the CGM 120, and an insulin infusion pump 140. In many embodiments, the communication between different components may not be direct. For example, a brain activity recorder may provide data to a CGM which in turn is provided to the controller rather than communicating with the controller directly. Indeed, as one of ordinary skill in the art would appreciate, any communications architecture can be used without departing from the scope or spirit of the disclosure herein.

In numerous embodiments, controllers process recorded brain activity to generate predictions regarding the patient's glucose levels. Controllers may provide predictions for only one of interstitial or blood glucose levels. In some embodiments both interstitial and blood glucose level predictions are computed. Further, controllers may be implemented using any of a variety of computing platforms. In various embodiments, the controller is a smart phone, a smart watch, a tablet computer, a personal computer, and/or any other personal wearable device. In some embodiments, the controller may be integrated into a medical device or a medical server system, e.g. a hospital computer network, or cloud medical system.

In various embodiments, insulin infusion pumps can variably infuse insulin as dictated by the controller. Further, other drugs rather than insulin may be provided via a similar infusion pump depending on the needs of the patient. As can be readily appreciated, many PGM systems may not include any infusion pumps if drug delivery is unadvisable for the particular patient. Similarly, PGMs may further include methods for delivering brain stimulation as an alternative treatment. In various embodiments, the brain activity recorder may also function as a brain stimulation device. Indeed, any number of different PGM system architectures can be used depending on the needs of a specific patient as appropriate to the requirements of specific applications.

Turning now to FIG. 2, a block diagram for any of the controllers herein is illustrated. Controller 200 includes a processor 210. In many embodiments, the processor is a logic circuit capable of executing instructions such as (but not limited to) a central processing unit (CPU), a graphics processing unit (GPU), a field programmable gate array (FPGA), an application-specific integrated circuit (ASIC), and/or any combination thereof. In many embodiments, more than one processor can be used. Controller 200 further includes an input/output (I/O) interface 220. The I/O interface can be used to communicate with different PGM system components and/or 3^{rd} party components such as (but not limited to) displays, speakers, CGMs, brain activity recorders, stimulation devices, infusion pumps, cellphones, medical devices, computers, and/or any other component via wired or wireless connections. Processor 210 may be any of other processors described herein, and vice versa.

Controller 200 further includes a memory 230. The memory 230 can be made of volatile memory, nonvolatile memory, or any combination thereof. The memory 230 contains a glucose management application 232. The glucose management application can direct the processor to carry out various PGM processes as described herein. In many embodiments, the memory 230 further contains brain activity data obtained from brain activity recorders. Brain activity data can describe brain activity as a signal or set of signals. In some embodiments, brain activity data includes waveforms recorded by sensor electrodes. In various embodiments, one or more waveforms are recorded for each electrode (“channel”). In a number of embodiments, the brain activity data describes the spectral profile of broadband brain activity. In a variety of embodiments, the glucose management application configures the processor to act as a multivariate decoder for brain activity data. As can be readily appreciated, controllers can be manufactured in different ways using similar computing components without departing from the scope or spirit of the disclosure. PGM processes are discussed in further detail below.

Predictive Glucose Management. PGM processes involve the collection and use of brain activity to predict future glucose levels of a patient. In numerous embodiments, treatment recommendations or the treatments themselves can be triggered by a prediction of hyper-or hypoglycemia in order to stabilize the glucose levels at a healthier range. Peripheral glucose levels tend to largely follow circadian dynamics and are strongly coherent to intracranial high frequency activity (HFA, 70-170 Hz) across multiple brain regions. As such, whole brain activity can be used in the predictive modeling process. In some embodiments, brain activity data from known glucose-sensors such as the hypothalamus, amygdala, and hippocampus are used instead of or in conjunction with brain activity from other regions and/or the whole brain.

In order to process data coming from one or more brain activity recorders, a machine learning model can be trained. In some embodiments, the training process is performed using data acquired from the patient on which the trained model will be used. In various embodiments, the model can be pre-trained on standardized data and training can be completed using patient data. In various embodiments, the model is continuously refined using predictions and subsequent validation as measured using a CGM. While linear models are often considered to be less predictive than more modem machine learning models, in many embodiments a linear model is sufficient for accurate prediction. However, in various embodiments, more complex predictive machine learning models can be used, such as (but not limited to) other types of regression models, neural networks, and others as appropriate to the requirements of specific applications.

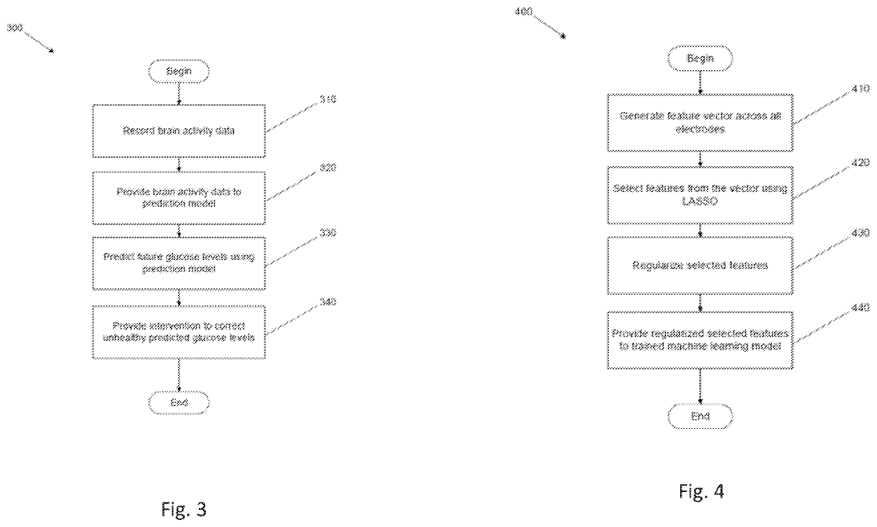

FIG. 3 illustrates an exemplary flow chart for an exemplary PGM process for predictively managing glucose levels. Process 300 includes recording (310) brain activity using a brain activity recorder, and providing (320) the brain activity data to a trained prediction model. The prediction model predicts (330) future glucose levels. In many embodiments, the certainty of the prediction may decrease the further in the future the prediction is made. In various embodiments, multiple predictions are provided at different time points, and only those above a predetermined confidence threshold as determined by a medical professional are used. In some embodiments, a hard limit is set on how far ahead the predictions are made for. If a hypo-and/or hyperglycemic state is predicted, a medical intervention is provided (340) to avoid the unhealthy dip or spike, respectively, in glucose levels. In various embodiments, the medical intervention is automatically provided, e.g. via control of an infusion pump and/or via brain stimulation. In various embodiments, a warning is provided to the patient and/or medical professional that an unhealthy glucose level is predicted. In some embodiments, confirmation is required before the medical intervention is provided.

While a particular process is illustrated in FIG. 3, as can readily be appreciated, various modifications can be made without departing from the scope or spirit of the disclosure. For example, pre-recorded brain activity data can be provided and used to make predictions. Further, interventions need not be recommended nor provided in all cases. In many situations, it is beneficial to merely have a warning.

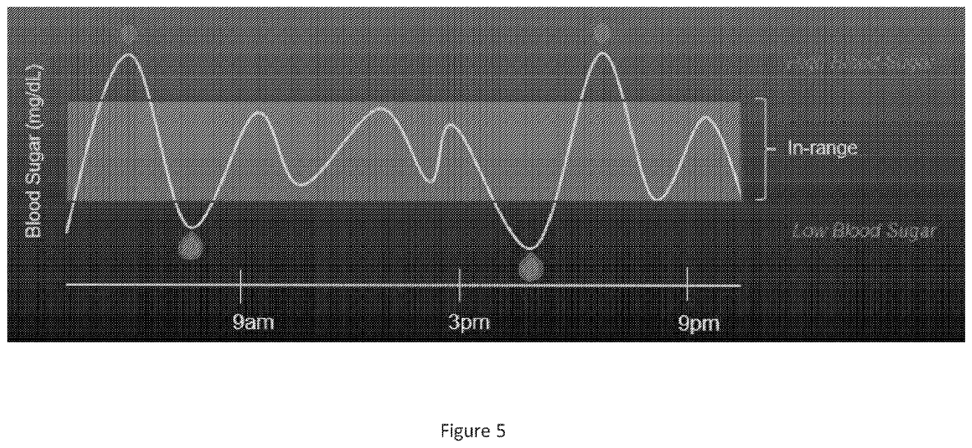

FIG. 4 is an exemplary flow chart of an exemplary PGM process for predicting glucose levels based on brain activity data. Process 400 includes generating (410) a feature vector across all electrode (or sensor) channels. In numerous embodiments, all frequency bands across all channels are flattened into a single feature vector. A Least absolute shrinkage and selection operator (LASSO) model is used to select (420) a subset of features from the feature vector for regularization (430). The regularized features are provided (440) to the trained machine learning model to produce one or more predictions. In numerous embodiments, a similar process is used to train the model using labeled training data from the patient and/or other patients. While specific machine learning models are discussed herein, many different machine learning models can be used without departing from the scope or spirit of the invention.

The disclosure below, including exemplary FIG. 5, describes systems and methods adapted to forecast future glucose levels non-invasively using a behind-the-ear scalp EEG sensor location. The entire disclosure of U.S. Pat. No. 11,020,035 is incorporated by reference herein for all purposes with respect to an exemplary wearable EEG sensor, any aspect of which may be incorporated into the EEG device (sensor) that is illustrated in FIG. 5 or any other wearable EEG sensor or sensing herein.

FIG. 5 illustrates an exemplary glucose forecasting system (“GFS”) including a plurality of components. The GFS includes a wearable EEG sensor (labeled “Mini EEG,”) which may include first and second paired electrodes. The EEG is adapted, sized and configured to be worn behind the ear and to measure EEG signals from the scalp. The GFS system may also optionally and not necessarily include an interstitial glucose-sensing insulin pump (e.g., a glucose monitor (such as a continuous glucose monitor) plus an insulin pump with infusion site) worn on the stomach. The GFS may also include an application or “App” stored on a smartphone or other personal device (or smart wearable device) that is adapted and configure to receive and analyze the EEG and glucose data continuously (and optionally as well as the delivered insulin) to produce a “forecast,” which may optionally be used so the insulin pump can deliver insulin. The App may optionally communicate with an online, secure patient management portal from which physicians remotely in clinic and patients at home or in clinic can see trends in the insulin delivery and EEG signal patterns, as well as their relationship.

The GFSs herein can be adapted and configured to forecast glucose levels and variations up to 6 hours prior to the actual change in level. Once a shift or change in glucose is forecasted, the App can optionally communicate a command to the insulin pump to deliver the adequate amount of needed insulin before extremes in glucose levels occur. The GFSs herein may optionally incorporate a glucose monitor (i.e., GFSs may not include a glucose monitor) until EEG-guided insulin titrations are further validated for the individual. GFSs may also rely on or utilize additional standard monitoring techniques, such as finger prick blood glucose monitoring. An exemplary benefit of GFSs over standard pumps is the ability to treat trends hours before they approach risky levels (either high or low glucose levels). Indeed, hypoglycemia is the most common problem seen with insulin-based therapies today. The GFSs herein provide for more safely titrating up and down predictive algorithms. Moreover, an optional closed-loop approach prevents dangerous out of range glucose levels, and may optionally even avoid having to monitor their own glucose levels. The GFSs herein may also be adapted to inform best dosing of long-acting insulin injections.

The GFSs herein may optionally be configured to provide information related to or about the glucose forecast. For example only, the App may be adapted to display at least a portion of the glucose forecast on a screen of a personal device (e.g., phone, wearable), and/or a predicted time or time period during which the forecast glucose levels will drop below a threshold or rise about a threshold.

FIG. 5 is a non-limiting example of a visual representation of forecasted blood glucose levels on a display of a device, such as a smartphone or other computing device. For example, an App may comprise a computer executable method stored in a non-transitory media (e.g. memory) that is adapted to, when executed by a processor, present forecasted future blood glucose levels, the method comprising: receiving as input extracranially-sensed EEG data or information indicative of extracranially-sensed EEG from a subject (from a behind the ear wearable EEG sensor); and causing a visual representation of the forecasted future blood glucose levels to be displayed on a display of a device. In exemplary FIG. 5, time is on the X-axis and the blood sugar levels on the Y-axis. The left side of the X-axis can be considered 3 am, where predicted levels that are out of range are shown. Visual indicators (e.g., the red icons) can indicate predicted peaks and valleys. The range may be user-adjustable on the display so the user can change the high or low levels for the range that is shown as “in-range.”

As mentioned above, existing closed-loop systems for detecting glucose levels and automatically delivering insulin are operating in real-time. They are reacting to peaks and dips in glucose as those changes happen. The GSFs described herein are adapted to sense EEG with a behind the ear wearable EEG sensor and forecast shifts in glucose levels hours before they occur, as well as optionally titrate the insulin based on this forecast before extremes occur in the glucose levels. Additionally, titrating insulin over time can dramatically decrease the risk of overcorrecting and causing symptomatic hypoglycemia, the most common disabling side effect of all current insulin-based treatments.

The disclosure that follows is related to the disclosure above in one or more ways. The description below may be incorporated and combined with the examples and embodiments above, and vice versa.

An aspect of this disclosure is related to forecasting blood glucose levels. The terms forecasting as used herein refers generally to predicting or determining future glucose levels or glucose state (interstitial and/or blood) and/or a risk of a future glycemic event of a subject (e.g., human), optionally an hour or more in advance. Forecasting may be performed to predict a future glycemic event, and optionally to prevent the forecasted glycemic event from occurring (examples of which are described herein). A glycemic event refers generally to a glucose level or state that is above or below certain levels (or within certain predefined parameters), such as above or below standard glucose levels.

Methods herein may optionally be adapted to provide forecasts that include one or more generalized risk levels or risk indicators for entering a future glycemic state within one or more certain periods of time in the future. For example only, a forecast may include a plurality of risk indicators, such as low risk, medium risk, and high risk. Optional risk indicators may optionally be visually presented on a display (on which a computer executable method is stored in a non-transitory media), such as a green light for time in the future where risk is low, a red light for time in the future where is risk is high, and a yellow light for time in the future where risk is medium.

Forecasting herein may include sensing EEG data (for example only, with optionally scalp and/or sub-scalp (subgaleal) electrodes) or receiving sensed EEG data, and analyzing the EEG data to forecast future blood glucose levels or states, and/or risk indicators. As described in more detail herein, however, it is conceivable that forecasting may occur without sensing any EEG data. For example, if a CGM is trained on sensed EEG data, existing CGMs may optionally be modified to be adapted to forecast future glucose levels and/or risk indicators based on the existing process of sensing ISG levels. Additionally and for example, one or more patient parameters (with or without EEG data) may be sensed/obtained and analyzed as part of the process of making the forecast, such as, without limitation, heart rate (HR), heart rate variability, skin conductance, blood pressure, body temperature, exercise level, etc. Any of these patient parameters may also be referred to herein as a user input.

In any of the methods herein, the optional EEG data may optionally be sensed continuously from a subject. In any of the methods herein, EEG data may optionally be sensed periodically from a subject. In some examples, EEG data may be sensed periodically (e.g., every 1 minute, every 5 minutes, etc.), and when a higher risk for a future glycemic event is forecasted, EEG data may be sensed continuously (or relatively more frequently) for some period of time.

Forecasting may optionally include providing a prediction of future blood glucose levels or risk indicators for some time period in the future, such as one or more hours, such as 1 hour, 2 hours, 3 hours, 4 hours, 5 hours, 6 hours, 7 hours, 8 hours, 9 hours, 10 hours, or more. This may be considered similar to a 7-day weather forecast.

The disclosure herein describes exemplary methods of forecasting and management. The disclosure also includes examples of devices and systems than may be adapted to perform one or more of the forecasting and/or management methods. It is intended that the devices and systems herein are exemplary, and other device and system arrangements may be able to perform or carry out one or more methods herein.

While some parts of the disclosure herein may be related to forecasting future blood glucose levels or risk indicators, an aspect of the disclosure herein is related to optional real-time (or near real time) blood glucose determination. The phrase “real-time” as used herein includes near real-time detection/determination as well, such as within five-fifteen minutes of an actual glucose level. Real-time blood glucose levels may optionally also be determined using sensed EEG data, optionally with one or more scalp or sub scalp electrodes. The systems herein may thus optionally be adapted to determine actual and/or forecasted blood glucose levels, or information indicative thereof. The systems herein may thus also be considered to be CGMs, similar to existing monitors.

One or more aspects of methods of forecasting herein may optionally be performed or executed on a personal device, such as a smartphone, tablet, smartwatch, etc., which may include one or more processors adapted to execute one or more computer executable methods/algorithms stored in a non-transitory media on the personal device. For example, a computer-executable application (an “App”) may, when executed by a processor, cause the processor to receive raw and/or processed EEG data (or information indicative of the raw or processed data) that has been sensed from the subject, and may be adapted to perform the forecasting process(es). In some alternatives, one or more processing steps may take place within the sensing device (e.g., scalp device or sub-scalp device), which is an example that methods herein may be performed in one or more different devices.

One or more aspects of a forecast and/or actual glucose levels or state may optionally be visually represented or presented on a display of a device (e.g., smartphone, tablet, smartwatch, electronic ophthalmic device such as a contact lens). For example only, an executable application (an “App”) may be adapted to visually present a risk indicator and/or forecasted glucose levels for the next three hours (or other number of hours), which may be updated (continuously or periodically) such that the forecast always includes predicted levels for the next three hours (or other number of hours). Additionally, for example only, an App may be adapted to visually present a specific time at which a glycemic event is forecasted to occur (e.g., 4:17 pm). Additionally, for example only, an App may be adapted to present a timer with a countdown indicating the time remaining before a forecasted glycemic event. The methods herein may optionally be adapted to provide a relatively short-term “forecast” of blood glucose measurements in advance (such as 1 hour in advance, 2 hours in advance, 3 hours in advance, etc.), and a longer-term “risk forecast” of blood glucose hours in advance (e.g., such as 10 hours in advance, 11 hours in advance, 12 hours in advance, etc.). Any of the methods herein (e.g., an App on a personal device) may optionally be adapted to communicate an actual and/or forecast of future glucose levels and/or risk indicators to a patient/care team, optionally to one or more different devices (an example of which is described herein as a Portal). Any of the methods herein (e.g., an App on a personal device) may optionally be adapted to provide a user/patient with a relatively longer (e.g., 10+hours) forecast of risk indicators, and optionally may be adapted to provide suggested (e.g., optimal) times of day to perform certain activities, such as exercise, eat, take medication based on the forecast. Any of the methods herein (e.g., an App on a personal device) may optionally be adapted to visually present (e.g., plot) an amount (e.g., percent) of time in optimal/preferred glucose range and time spent out of the optimal/preferred range, wherein the range may be adjustable and/or personalized, optionally wherein the method (e.g. App) is adapted to allow for personalized adjustment and setting of the range via interaction with a display of the personal device. One aspect of the disclosure is a computer executable method that is adapted to present interactive features (e.g., icons on a screen, up/down arrows, audible instructions, etc.) that allow a user (patient and/or care team member(s) to adjust a range of glucose levels.

Any of the methods herein may optionally be adapted to provide or initiate an alert when an existing glycemic event or state has been detected and/or when a future glycemic event has been forecast. For example, once a future glycemic event has been forecast any of the alerts herein may be triggered (audible alert, text alert, email, alert to patient and/or caregiver/care team, etc.). Any of the methods herein (e.g., an App on a personal device) may optionally be adapted to provide or initiate an alert when glucose is trending out of range and/or is currently out of range. Any of the methods herein (e.g., an App on a personal device) may optionally be adapted to provide or initiate a relatively high frequency pitch (47-65 Hz) alert when glucose is trending out of range, which may alert a service animal to the forecast or detected glycemic event. One aspect of this disclosure is an executable method that is adapted to provide or initiate a relatively high frequency pitch (e.g. 47-65 Hz) alert when a medical event has been detected and/or forecast, such as a glucose level trending out of range, a seizure is detected or predicted, or loss of consciousness predicted, etc.

One or more aspects of the actual and/or forecasted blood glucose levels (or information indicative thereof or related thereto) may optionally be communicated, optionally to the subject and/or third party (e.g., caregiver, family member, etc.). One or more aspects of the actual and/or forecasted blood glucose levels or states may optionally be communicated to one or more devices, which may be the same as or different than the device that determines the actual and/or forecast blood glucose information.

The methods and systems herein may optionally be adapted to continuously stream real-time EEG data to a different device, such as any of the personal devices herein (smartphone, watch, etc.). The Apps herein may thus be receiving continuous or near-continuous real-time EEG data that is being sensed from the patient, and using the continuously streamed real-time EEG data to make the forecasts.

The methods and systems herein may optionally be adapted to receive one or more non-EEG patient parameters, such as skin conductance, heart rate, blood pressure, etc., any of which may be inputs to the forecasting and/or detection methods herein. Any of the methods herein (e.g., an App on a personal device) may optionally be adapted to receive one or more inputs, optionally from one or more of a patient or caregiver. For example, any of the methods herein (e.g., an App on a personal device) may optionally be adapted to allow for user-input of information such as insulin administration/dose, medication timing, food, exercise, stress, sleep and illness, for example. Any of the methods herein (e.g., an App on a personal device) may optionally be adapted to allow a user (patient and/or caregiver) to mark or indicate events on raw EEG tracings (or processed EEG data).

Part of this disclosure includes methods that are adapted to use one or more inputs to refine or train (e.g., increase the accuracy thereof) any of the forecasting algorithms herein, and optionally the inputs may alternatively or in addition to refine or train any of the CGMs herein. For example, and without limitation, inputs include, without limitation, inputs that are input manually via a user and/or inputs received from other devices (e.g., pumps, glucose meters, and/or CGMs). For example, Apps herein may be adapted to receive information from a glucose meter and/or CGM (e.g., blood glucose readings/values) and use that information to refine a forecasting algorithm. A user may optionally input blood glucose values. Inputs in this context may further include, without limitation, what food was consumed and/or when they consumed the food; when insulin was administered and/or a dose, or any other input that is related to the life of the user that may be able to refine a forecasting method.

There may be (but not necessarily) some patient variability in the forecasting. For example only, there may be some variability in the time period during which accurate forecasts can be made. For example only, for some patients it may be possible to most accurately forecast 8 hours in advance, while for some patients it may be possible to most accurately forecast 2 hours in advance. The systems and methods herein may thus have some personalization for individual patients.

Additionally, for example, there may be some variability in when it is possible to provide a peak or optimal forecast for a patient. For example, a first patient may have optimal forecasting 2-3 hours before a glycemic event (or not before 3 hours before an event, for example), but a second patient may have optimal forecasting 4-5 hours before an event (or not before 5 hours before an event, for example). The systems and methods herein may thus have some personalization for individual patients.

An aspect of this disclosure is related to the management of blood glucose levels. In some examples, management of blood glucose levels includes preventing a glycemic event, such as a hypoglycemic event or a hyperglycemic event. In some examples, management of blood glucose levels optionally also includes ceasing or minimizing the severity of an existing glycemic event or state. The methods of management herein may include guiding medication and insulin (long/short acting) dosage based on glucose forecast.

It is understood that some concepts above such as Alerts and Outputs (optionally provided by an App) may also considered part of an overall approach to Managing blood glucose levels. For example, communicating insulin and medication needs to a user based on real-time and forecasted blood glucose may be considered part of Management. Additionally, for example only, automated meal and exercise recommendations (optionally communicated via an App) to a subject may be considered part of the Management.

It is understood that aspects of this disclosure that are related to forecasting/detecting blood glucose levels may or may not be incorporated with aspects of this disclosure related to management of blood glucose levels.

A merely exemplary method of management includes closed loop functionality, and optionally may include a pump that may be adapted to deliver one or more agents (e.g., glucagon, insulin, etc.). For example, one aspect of the disclosure is a method of controlling future glucose levels in a patient in response to measuring/sensing EEG signals and/or patterns (although other inputs may be used as part of the controlling process-e.g., HR, skin conductance, etc.). In this exemplary aspect, controlling may include delivering insulin or glucagon to a patient before a glucose level deviates from a desired range/limit. In this aspect, controlling may include delivering insulin to a patient at a time when the glucose level is still in a safe range (e.g., 70-180, 80-170, etc.). In this aspect, controlling may include maintaining glucose levels within a safe/preferred range. In this aspect, controlling may include delivering an agent (e.g. insulin) before the time at which glucose levels are predicted to deviate from a safe range. In this aspect, controlling may include delivering a particular dose of insulin based on the time the glucose levels are predicted to deviate from a safe range. In this aspect, controlling may include delivering a dose of insulin that is different than the dose that would be administered in response to a real time glucose level monitoring process, such as via a CGM.

The systems herein may optionally include a multiple-chamber pump, such as a dual chamber pump adapted to deliver insulin and glucagon to manage future glucose levels.

The systems herein may be adapted and configured to integrate with Bluetooth-enabled insulin pumps (e.g. Omnipod).

Patients in an ICU setting have their blood glucose levels measured periodically, and typically with a finger prick and tested on a glucose meter, a process which may take 10 minutes to complete. Each result is thus roughly 10 minutes delayed. Being able to forecast blood glucose levels could be incredibly helpful in ICU setting, both from a patient care as well as hospital resource management perspective. ICU patients typically already have EEG electrodes attached, and thus EEG can be sensed and analyzed to forecast (or help forecast) future blood glucose levels. Any of the methods and systems described herein may thus advantageously be used in an ICU setting. Currently, insulin drips are typically provided to ICU patients, with a sliding scale of dosage as part of the current care.

Any of the systems and methods herein may optionally be adapted to deliver insulin when a hyperglycemic event is forecast. Any of the systems and methods herein may optionally be adapted to deliver glucagon (e.g., intranasally; with a pump, etc.) when a hypoglycemic event is forecast. As an example of Management of hypoglycemia herein, in any of the methods herein, an alert or recommendation to consume, for example, a high sugar drink.

Any of the systems and methods herein may include one or more of oral medicine delivery, subcutaneous delivery, or delivery via a pump.

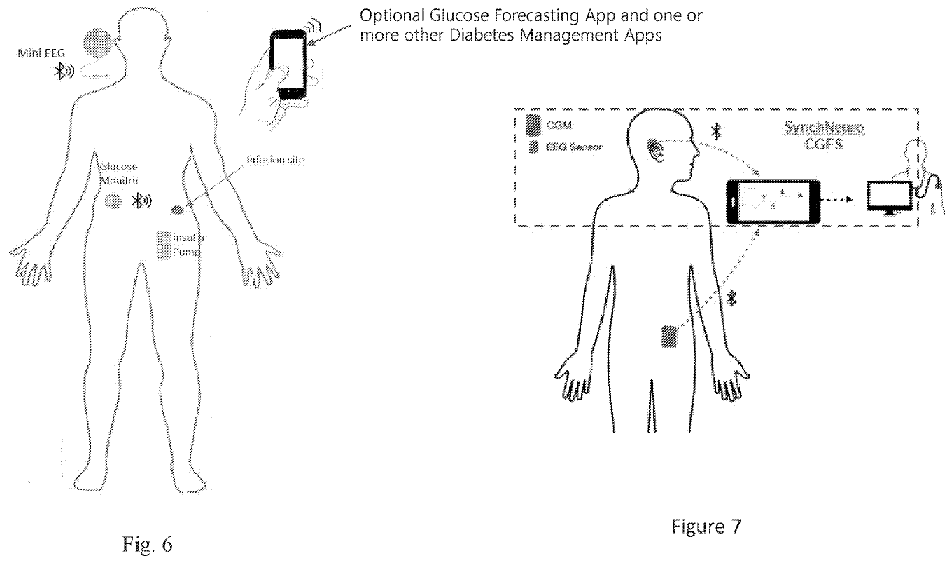

As set forth, the disclosure includes exemplary systems, but it is understood that systems with different arrangements may be adapted to perform many if not all of the methods herein. FIG. 6 provides a merely exemplary, non-limiting illustration of a glucose forecasting system, and which may optionally not include all of the components shown. The “mini EEG” in FIG. 6 is merely representative of an exemplary wearable sensor. The “Glucose Monitor” in FIG. 6 is merely representative of an exemplary CGM. The smartphone shown is merely representative of a personal device on which an App may be stored. An optional pump is also illustrated. Wireless capabilities (e.g., Bluetooth) are also illustrated.

In a merely exemplary example, a system herein may be a continuous glucose forecasting system, in which one or more patient parameters (e.g., EEG) are continuously monitored. FIG. 7 illustrate an exemplary continuous glucose forecasting system (CGFS). In alternative systems, the monitoring may be periodic or a combination of continuous and periodic.

Optionally, the systems herein include one or more wearable, rechargeable sensors (which may be referred to as a wearable sensing device or sensing device), such as a plurality of (e.g., two) electrodes worn optionally behind the ear. The sensing devices herein may optionally have Bluetooth or other wireless communication capabilities. The sensing devices herein may be adapted to measure EEG signals continuously, near-continuously, and/or periodically. U.S. Pat. No. 11,020,035 is a mere example of a wearable EEG monitoring/recording system, any features of which may be incorporated into any of the wearable sensing devices, systems, and/or methods of use herein.

Wearable sensing devices herein may or may not have storage capabilities. Wearable sensing devices herein may or may not have signal processing/analyzing capabilities.

Any of the wearable sensing devices herein may be affixed to a scalp of the subject, and in other examples they may be in a sub scalp (subgaleal) form.

Any of the wearable sensing devices herein may include a micro-needle array, which can be adapted to sense ISF (similar to CGMs). A needle array can be positioned such that the needles extend into the skin.

As a mere example, the wearable sensors herein may include one or more of: being completely non-invasive; a multi-electrode (e.g. two; single channel) patch, adapted to be worn behind the ear for several (e.g., 30+) days in sequence; silicon (patch) and stainless steel (“dry” electrodes); sticker (changeable) may hold the patch in place on skin; may include a memory chip for at least temporary storage of data (e.g., if a personal device is not nearby, the sensing device may need to be able to store data until it can be transmitted to a personal device; bluetooth communication capabilities; waterproof; rechargeable; an optional microneedle/microarray integrated into the patch for ISG measurements.

Optionally, the systems herein include a personal device (e.g., smartphone, watch, tablet) that optionally has an App stored thereon. The App may be accessed by the subject or caregiver. An App is optionally adapted to receive/gather data from the one or more wearable sensors, process the received information (to some extent) and can optionally display actual and forecasted blood glucose measurements or risk indicators to the user, which is described in more detail above. An App is optionally also adapted to display trends of past blood glucose measurements and user insights on blood glucose levels. An App is optionally adapted to store EEG data (raw and/or processed), and optionally until is transferred to a different device, such as an optional online portal described below. A personal device may have more data storage, so it may be better suited to store more data, allowing a wearable sensor to have a smaller form factor.

Any of the Apps on a personal device herein may be adapted to perform any of the methods herein, or cause a processor to perform the computer executable instructions of the App (e.g., executable methods, such as forecasting).

Optionally, the systems herein include an online portal, which is optionally available to the subject and any other individual approved by the user (e.g. physician, caregiver, care team, family member, etc.). The portal may be adapted to display any of the information or data described herein, including any historical data on blood glucose trends and user insights on blood glucose levels. The portal may optionally be adapted to display raw EEG signals. The online portal may have signal processing and/or analyzing capabilities as well.

As a mere example, an online portal may comprise any of the following functionality or capabilities: communicates the actual and forecast of future glucose levels to patient/care team; communicates a trend of past glucose measurements; communicates insulin and medication information to user based on real-time and forecasted blood glucose: provides alerts when glucose is trending out of range or is out of range: provides user with long-term (e.g., 10+hour) forecast of “risk states” to suggest optimal times of day to exercise, eat meals, and take medication based on the glucose forecast; adapted to displays outside insulin pump, glucose monitor, and App information from Bluetooth linked systems; adapted to displays raw EEG tracing, with user-input markers; can display any other related health data.

One aspect of the disclosure is related to training forecasting methods or models (e.g., computer executable forecasting methods or models) for forecasting a future glucose state of subjects (e.g., a future blood glucose level or a risk indicator for a future blood glucose level). The training may include sensing one or more patient parameters (e.g. EEG signals) from one or more subjects, optionally non-invasively, and optionally from a scalp location. The training may optionally include processing the sensed EEG signals. The training may include sensing real-time or near real-time blood glucose levels from one or more subjects (which may be interstitial glucose levels represented of blood glucose levels), or information indicative of real-time or near real-time blood glucose levels; and then creating an association with the or more sensed parameters (e.g. EEG signals) and the sensed real-time or near real-time blood glucose levels or information that is indicative of real-time or near real-time blood glucose levels. In this manner, a forecasting method can be trained to forecast in advance when future glycemic events will or will likely occur (with at least some degree of accuracy).

Any of methods/algorithms herein may be trained on one or more of normal, hyperglycemia (e.g., diabetes; hyperglycemia ICU, sepsis, traumatic brain energy; diabetic ketoacidosis) or hypoglycemia states.

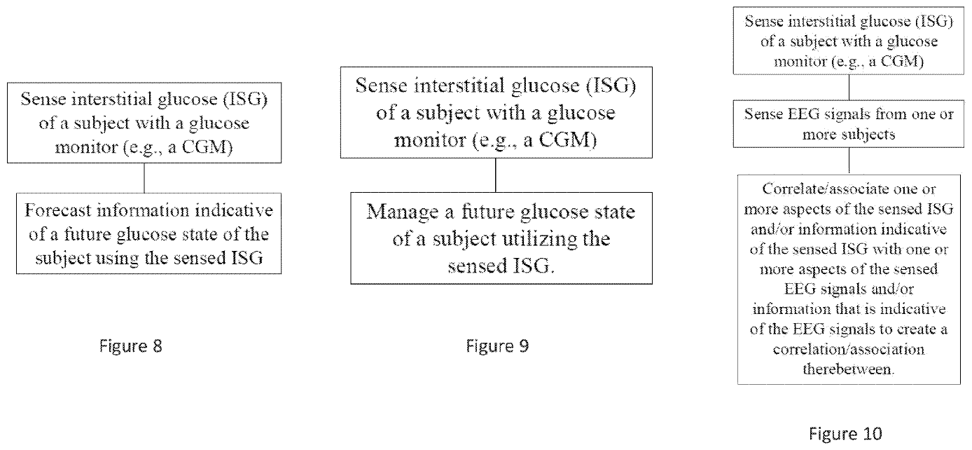

The systems, devices, and methods herein may be adapted to be incorporated with glucose monitors, such as CGMs, and/or their methods of use. For example, existing CGMs may be modified and adapted to incorporate sensed EEG data (for example only, any sensing concepts/methods in U.S. Pat. No. 6,572,542 and/or U.S. Pat. No. 8,118,741) and/or forecasting concepts herein to improve performance. For example only, CGM sensed data can be analyzed with patient EEG data, and the predictive EEG data can train the interstitial glucose (ISG) data, so that the CGM may then be adapted to use ISG readings to better predict future blood glucose states, exemplary method steps of which are shown in FIG. 8, and which may be combined with any other suitable method step herein. For example, a certain pattern of EEG-trained ISG data (readings well before an impending event) can then be used to predict a future glycemic event. It is thus understood that any existing CGM may be modified and adapted to incorporate any of the features or methods herein. For example, a CGM can be adapted to communicate with an App and make an alert that a subject should prepare to drink a sugary drink in a certain period of time, such as 1 hour in the future, or that a hypoglycemic event is likely to occur 2.5 hours in the future. Additionally, for example, a CGM could be modified to deliver insulin at a time much earlier than with previous technologies, and could deliver longer lasting insulin well in advance of a hyperglycemic event. FIG. 9illustrates merely exemplary method steps in which sensed ISG can be used to manage a future glucose state of a subject.

FIG. 10 illustrates merely exemplary steps that may be included in a method of training ISG data with EEG data, which may be performed to allow a glucose monitor to sense ISG and 1) forecast information indicative of a future glucose state (e.g., FIG. 8) and/or 2) facilitate the management of a future glucose state (e.g., FIG. 8).

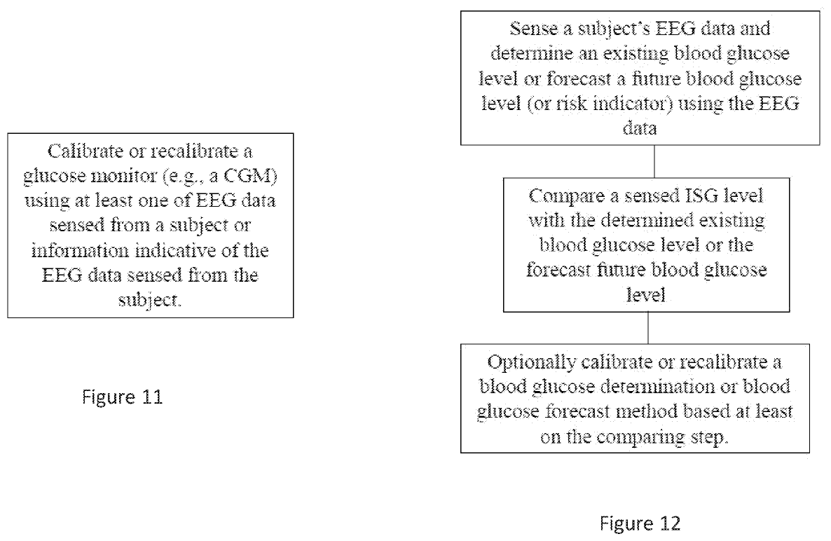

Additionally, and only for example, any of the EEG data and methods herein may optionally be used to help calibrate and/or recalibrate glucose monitors (e.g., CGMs) (which need recalibrating over time), which could avoid the need to use glucose meters and finger pricks to re-calibrate glucose monitors such as CGMs. FIG. 11 illustrates a merely exemplary method of calibrating or recalibrating a glucose monitor, optionally a CGM, comprising: calibrating or recalibrating a glucose monitor using at least one of EEG data sensed from the subject or information indicative of the EEG data sensed from the subject. Additionally, glucose monitors (e.g., CGMs) and/or glucose meters may similarly be used to calibrate any of the EEG forecasting methods (e.g., algorithms) herein, an example of which is shown in FIG. 12.

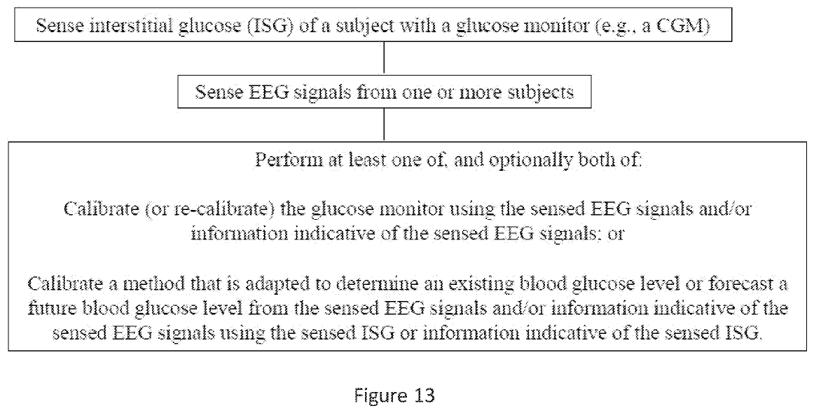

One aspect of the disclosure is an optional bi-directional calibration method and/or system, and example of which is shown in FIG. 13. In one example, a bi-directional calibration method may include sensing interstitial glucose of a subject with a glucose monitor, optionally a CGM; sensing EEG signals from one or more subjects, optionally with any of the wearable devices herein; and performing at least one of, and optionally both of: calibrating (or re-calibrating) the glucose monitor using the sensed EEG signals and/or information indicative of the sensed EEG signals; or calibrating a method that is adapted to determine an existing blood glucose level or forecast a future blood glucose level from the sensed EEG signals and/or information indicative of the sensed EEG signals using the sensed ISG or information indicative of the sensed ISG.

Additionally, as mentioned above, thresholds for CGMs are currently reset (when they need to be reset) at an office visit. Sensing EEG data its forecasting aspect may even allow for resetting thresholds without requiring an office visit, such as by using an online portal herein.

Additionally, CGMs can be used in conjunction with any of the other heath data/parameters (e.g., user inputs) herein (e.g., heart rate, blood pressure, skin conductance-which can be sensed easily by existing devices such as smartwatches, fitbits, etc.) to optionally predict future glucose states.

Exemplary CGMs, features and methods of use of which may be incorporated herein include those by Dexcom (e.g., G6 CGM System), Medtronic (e.g., Guardian™ Connect), Abbott (e.g., any FreeStyle Libre), and the Eversense® E3 CGM.

Exemplary Glucose Meters (glucometers), features and methods of use of which may be incorporated herein: LifeScan OneTouch®, Accu-Chek®, and FreeStyle Lite by Abbott.

Exemplary Insulin pumps, features and methods of use of which may be incorporated herein: Medtronic Minimed™, Pumps from Tandem, and Omnipod® pumps.

Any of the features from any of the examples or embodiments herein may be combined with any other feature unless it is expressly stated otherwise herein. For example, any of the methods herein may or may not be performed by a system or device.

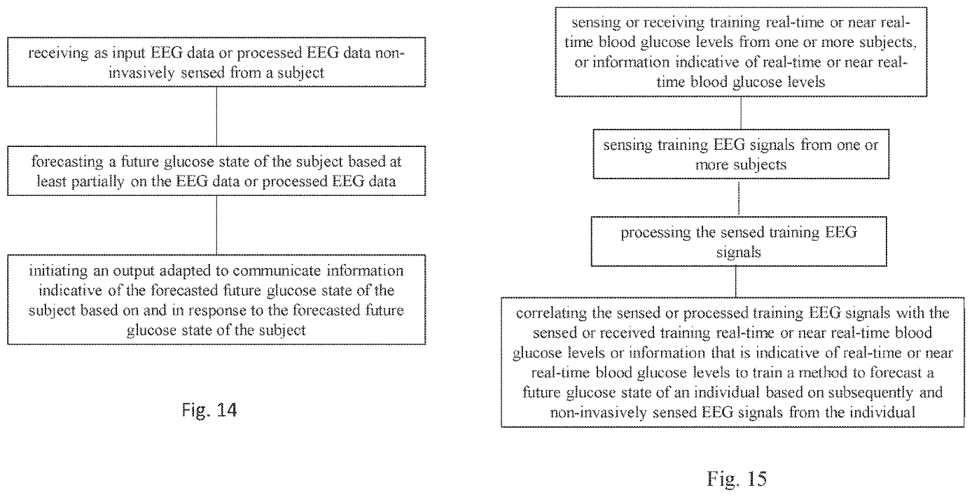

FIG. 14 illustrates an exemplary method that includes the steps as shown. Any of the methods herein may include receiving as input EEG data or processed EEG data non-invasively sensed from a subject; forecasting a future glucose state of the subject based at least partially on the EEG data or processed EEG data; and initiating an output adapted to communicate information indicative of the forecasted future glucose state of the subject based on and in response to the forecasted future glucose state of the subject, as shown in FIG. 14.

FIG. 15 illustrates an exemplary training method that includes the steps as shown. Any of the training methods herein may include sensing or receiving training real-time or near real-time blood glucose levels from one or more subjects, or information indicative of real-time or near real-time blood glucose levels; sensing training EEG signals from one or more subjects; processing the sensed training EEG signals; and correlating the sensed or processed training EEG signals with the sensed or received training real-time or near real-time blood glucose levels or information that is indicative of real-time or near real-time blood glucose levels to train a method to forecast a future glucose state of an individual based on subsequently and non-invasively sensed EEG signals from the individual, as shown in FIG. 15.

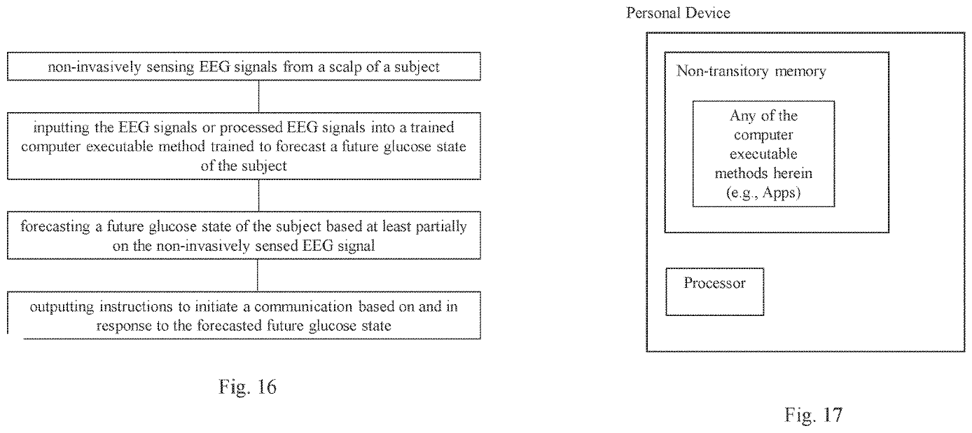

FIG. 16 illustrates an exemplary method that includes the steps as shown. Any of the methods herein may include non-invasively sensing EEG signals from a scalp of a subject; inputting the EEG signals or processed EEG signals into a trained computer executable method trained to forecast a future glucose state of the subject; forecasting a future glucose state of the subject based at least partially on the non-invasively sensed EEG signal; and outputting instructions to initiate a communication based on and in response to the forecasted future glucose state, as shown.

FIG. 17 illustrates exemplary components of a personal device, which may be any personal device herein, and which may include additional components therein (such as, without limitation, any of the exemplary hardware components in FIG. 19). The personal devices herein may include one or more processors (shown as a single “processor”), and one or more non-transitory memory or media. The media has stored therein any of the computer executable methods herein (e.g., an App).

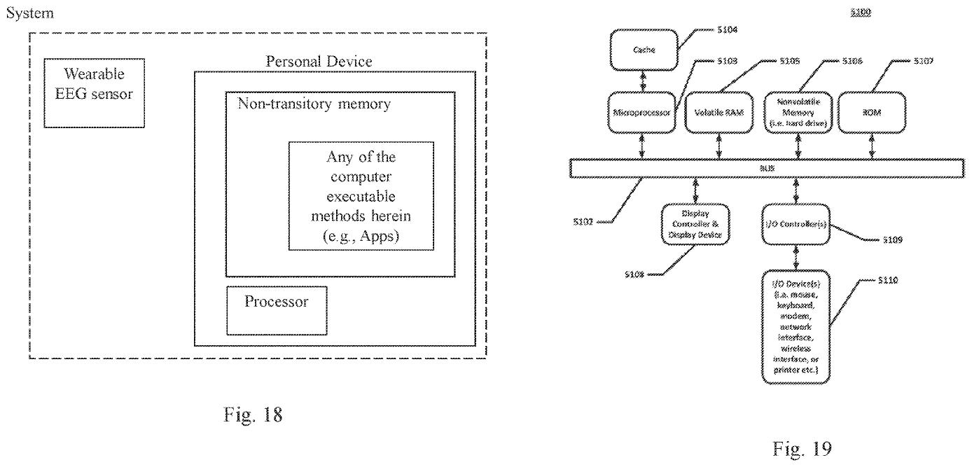

FIG. 18 illustrates an exemplary System, which includes at least one wearable EEG sensor (such as any of the behind the ear sensors and/or single channel EEG sensors herein), and a personal device. The description of the personal device from FIG. 17 is fully incorporated by reference herein into the description of FIG. 18. The system of FIG. 18 may be any of the systems herein, and which may include other components, such as any of the components herein.

FIG. 19 illustrates a mere example of one or more components that may be included in any of the sensors and/or computing devices herein (such as personal devices herein). The reference labels are understood to refer to the textual description of the component shown in FIG. 19.

Even if not specifically indicated, one or more methods or techniques described in this disclosure (e.g., any of the computer executable methods adapted to be executed by a processor which may be stored in non-transitory media) may be implemented, at least in part, in hardware, software, firmware or any combination thereof. For example, various aspects of the techniques or components may be implemented within one or more processors, including one or more microprocessors, digital signal processors (DSPs), application specific integrated circuits (ASICs), field programmable gate arrays (FPGAs), programmable logic circuitry, or the like, either alone or in any suitable combination. The term “processor” or “processing circuitry” may generally refer to any of the foregoing circuitry, alone or in combination with other circuitry, or any other equivalent circuitry.

Such hardware, software, or firmware may be implemented within one device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as random access memory (RAM), read only memory (ROM), non-volatile RAM (NVRAM), electrically erasable programmable ROM (EEPROM), Flash memory, and the like. The instructions may be executed by a processor to support one or more aspects of the functionality described in this disclosure.

1. A method of forecasting a future glucose state of a subject, comprising:
non-invasively sensing EEG signals from a behind the ear location on a scalp of a subject;
inputting the EEG signals or processed EEG signals into a trained computer executable method trained to forecast a future glucose state of the subject;
forecasting a future glucose state of the subject based at least partially on the non-invasively sensed EEG signal; and
outputting instructions to initiate a communication based on and in response to the forecasted future glucose state.

2. The method of claim 1, wherein non-invasively sensing EEG signals comprises sensing EEG signals with a device including a plurality of sensing electrodes, wherein the plurality of sensing electrodes includes only first and second sensing electrodes.

3. The method of claim 1, further comprising wirelessly communicating sensed EEG signals to a personal device, wherein the computer executable method is stored on the personal device, and wherein the forecasting step occurs on the personal device.

4. The method of claim 1, wherein outputting instructions to initiate a communication comprises outputting instructions to initiate a communication in the form of information displayed on a display.

5. The method of claim 4, wherein the display is a display on a personal device.

6. The method of claim 4, wherein the communication comprises causing a personal device to visually present on a display information that is indicative of the forecasted future glucose state.

7. The method of claim 1, wherein forecasting a future glucose state of the subject comprises forecasting future one or more interstitial glucose levels.

8. The method of claim 1, wherein outputting instructions to initiate a communication comprises outputting instructions to initiate a communication that causes the delivery of insulin to the subject.

9. A computer-executable method, stored in a non-transitory media, adapted to, when executed by a processor, cause the performance of:
receiving as input EEG data or processed EEG data non-invasively sensed from a behind the ear location on a scalp of a subject;
forecasting a future glucose state of the subject based at least partially on the EEG data or processed EEG data; and
initiating an output adapted to communicate information indicative of the forecasted future glucose state of the subject based on and in response to the forecasted future glucose state of the subject.

10. The method of claim 9, wherein the receiving step comprises receiving as input EEG data or processed EEG data non-invasively sensed from a wearable device at the behind the ear location, the wearable device including a plurality of electrodes that include no more than first and second sensing electrodes.

11. The method of claim 9, further comprising wirelessly communicating sensed EEG signals to a personal device, wherein the computer executable method is stored on the personal device, and wherein the forecasting step occurs on the personal device.

12. The method of claim 9, wherein initiating the output adapted to communicate information comprises initiating an output that causes the information to be communicated to a personal device.

13. The method of claim 12, wherein the information is visually represented on a display of the personal device.

14. The method of claim 9, wherein forecasting a future glucose state of the subject comprises forecasting future interstitial glucose levels.

15. The method of claim 9, wherein initiating an output adapted to communicate information comprises initiating an output that is adapted to communicate information that causes the delivery of insulin to the subject.

16. A non-transitory, computer-readable storage media with instructions stored thereon and executable by a processor to perform a method, the method comprising:
receiving as input EEG data or processed EEG data non-invasively sensed from a behind the ear location on a scalp of a subject;
forecasting a future glucose state of the subject based at least partially on the EEG data or processed EEG data; and
initiating an output adapted to communicate information indicative of the forecasted future glucose state of the subject based on and in response to the forecasted future glucose state of the subject.

17. The media of claim 16, further comprising a personal device on which the non-transitory, computer-readable storage media is stored, the device further comprising the processor.

18. The media of claim 16, wherein the personal device comprises a display, and wherein initiating an output adapted to communicate information comprises initiating an output that causes the information to be communicated to the personal device.

19. The method of claim 18, wherein the information is visually represented on a display of the personal device.

20. The media of claim 16, wherein forecasting a future glucose state of the subject comprises forecasting future interstitial glucose levels.

21. The media of claim 16, wherein initiating an output adapted to communicate information comprises initiating an output that is adapted to communicate information that causes the delivery of insulin to the subject.

22.-42. (canceled)
